(19) United States
(12) Patent Application Publication
(10) Pub. No.: US 2013/0150881 A1
(43) Pub. Date: Jun. 13, 2013

(54) VALVULOPLASTY DEVICE

(71) Applicant: Boston Scientific Scimd, Inc., Maple Grove, MN (US)

(72) Inventors: Huisun Wang, Maple Grove, MN (US); James M. Anderson, Fridley, MN (US); Jan Weber, Maastricht (NL)

(73) Assignee: Boston Scientific Scimed, Inc., Maple Grove, MN (US)

(21) Appl. No.: 13/712,405

(22) Filed: Dec. 12, 2012

Related U.S. Application Data
(60) Provisional application No. 61/570,011, filed on Dec. 13, 2011.

(51) Int. Cl. A61M 29/02

(52) U.S. Cl. CPC A61M 29/02
USPC 606/194

(57) ABSTRACT

A medical device may include an elongate shaft having a plurality of elongated balloons disposed at a distal end including a first balloon, a second balloon, and a third balloon, a manifold disposed at a proximal end of the elongate shaft in fluid communication with the plurality of balloons, and a distal tip disposed distal of the plurality of balloons, the distal end of each balloon being joined together by the distal tip. A medical device may include an elongate shaft having a plurality of elongated balloons disposed at a distal end including a first opposing pair and a second opposing pair, a manifold disposed at a proximal end of the elongate shaft in fluid communication with the plurality of balloons, and a distal tip disposed distal of the plurality of balloons, the distal end of each balloon being joined together by the distal tip.

### CROSS-REFERENCE TO RELATED APPLICATIONS

None.

### TECHNICAL FIELD

The disclosure relates generally to medical devices and more particularly to medical devices that are adapted for use in repairing heart valves.

### BACKGROUND

Valve stenosis or calcification is a frequent expression of valvular heart disease, and may often be a leading indicator for balloon valvuloplasty and/or valve replacement therapy in Europe and the United States. The prevalence of valve stenosis tends to increase in older population groups. In some cases, traditional valve replacement surgery is not suitable for patients with higher surgical risk factors. Alternate therapies, such as balloon valvuloplasty, may be beneficial in improving the lifestyle of patients suffering from valve stenosis.

A continuing need exists for improved balloon valvuloplasty devices and methods as an alternative to traditional valve replacement surgery.

### SUMMARY

A medical device for valvuloplasty of a heart valve may include an elongate shaft having a plurality of elongated balloons disposed at a distal end thereof, each elongated balloon including a proximal end and a distal end, wherein the plurality of elongated balloons further comprises a first balloon, a second balloon, and a third balloon, a manifold disposed at a proximal end of the elongate shaft, the manifold in fluid communication with the plurality of elongated balloons, and a distal tip disposed distal of the plurality of elongated balloons, the distal end of each elongated balloon being joined together by the distal tip, wherein the plurality of elongated balloons is configured to selectively expand from a collapsed configuration to an inflated configuration.

A medical device for valvuloplasty of a heart valve may include an elongate shaft having a plurality of elongated balloons disposed at a distal end thereof, each elongated balloon including a proximal end and a distal end, wherein the plurality of elongated balloons further comprises a first balloon and a second balloon arranged as a first opposing pair, and a third balloon and a fourth balloon arranged as a second opposing pair, a manifold disposed at a proximal end of the elongate shaft, the manifold in fluid communication with the plurality of elongated balloons, and a distal tip disposed distal of the plurality of elongated balloons, the distal end of each elongated balloon being joined together by the distal tip, wherein each of the plurality of elongated balloons is configured to selectively actuate between a collapsed configuration and an inflated configuration.

Although discussed with specific reference to use within the coronary vasculature of a patient, for example to repair a heart valve, medical devices and methods of use in accordance with the disclosure can be adapted and configured for use in other parts of the anatomy, such as the digestive system, the respiratory system, or other parts of the anatomy of a patient.

### BRIEF DESCRIPTION OF THE FIGURES

FIG. 1A is a schematic partial cross-sectional view of a tri-leaflet heart valve;

FIG. 1B is a schematic partial cross-sectional view of a bi-leaflet heart valve;

FIG. 2A is a schematic partial cross-sectional view of a diseased tri-leaflet heart valve with asymmetric calcification;

FIG. 2B is a schematic partial cross-sectional view of a diseased tri-leaflet heart valve with asymmetric calcification;

FIG. 3A is a schematic partial cross-sectional view of the diseased tri-leaflet heart valve of FIG. 2A being treated with single-balloon valvuloplasty;

FIG. 3B is a schematic partial cross-sectional view of the diseased tri-leaflet heart valve of FIG. 2B being treated with single-balloon valvuloplasty;

FIG. 4 is a side view of an example medical device;

FIG. 5 is a partial side view of an example medical device;

FIG. 6A is a schematic partial cross-sectional view of the example medical device of FIG. 5;

FIG. 6B is a schematic partial cross-sectional view of the example medical device of FIG. 6A including a cutting element;

FIG. 7A is schematic partial cross-sectional view of the example medical device of FIG. 6A in a partially inflated condition;

FIG. 7B is schematic partial cross-sectional view of the example medical device of FIG. 6B in a partially inflated condition;

FIG. 8A is schematic partial cross-sectional view of the example medical device of FIG. 7A in a fully inflated condition;

FIG. 8B is schematic partial cross-sectional view of the example medical device of FIG. 7B in a fully inflated condition;

FIG. 9 is a partial side view of an example medical device;

FIG. 10 is a schematic partial cross-sectional view of the example medical device of FIG. 9;

FIGS. 11-13 show schematic partial cross-sectional views of the example medical device of FIG. 9 in differing stages of inflation; and

FIG. 14 is a partial side view of an example medical device.

While the invention is amenable to various modifications and alternative forms, specifics thereof have been shown by way of example in the drawings and will be described in greater detail below. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

The terms “upstream” and “downstream” refer to a position or location relative to the direction of blood flow through a particular element or location, such as a vessel (i.e., the aorta), a heart valve (i.e., the aortic valve), and the like.

With respect to a heart valve, the term “free margin” refers to an edge or portion of a valve leaflet which is free to move relative to the valve annulus. The “leaflet intersection” refers to a region or regions where the free margins of two adjacent valve leaflets come together or abut each other when the valve is in a closed condition.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout several views. The detailed description and drawings are intended to illustrate but not limit the claimed invention.

A human heart includes several different heart valves, including aortic, pulmonary, mitral, and tricuspid valves, which actuate between a closed condition and an open condition to control the flow of blood to and from the heart. Valve leaflets that are free of disease or stenosis are generally very flexible and cooperate to function as a one-way or check valve permitting blood to flow downstream when open and preventing blood from flowing back through the valve in an upstream direction when closed. Over time, a heart valve may become obstructed, narrowed, and/or less flexible (i.e., stenosed) due to hardening, calcium deposition (i.e. calcification), or other factors, thereby reducing the flow of blood through the valve and/or increasing the pressure within the chambers of the heart as the heart attempts to pump the blood through the vasculature. In some cases, valve stenosis may result in one or more valve leaflets becoming hardened and/or fused together by calcium deposits, thereby reducing the flexibility and effectiveness of the valve leaflet(s). In some cases, a diseased or stenosed heart valve may become so hardened that the valve leaflets can no longer achieve a fully open (and/or in some cases a fully closed) condition. One traditional treatment method is valve replacement, where the stenosed valve is removed and a replacement tissue or mechanical valve is implanted via open heart surgery. For some patients, an alternative to valve replacement may be valve repair, where the native heart valve is repaired percutaneously, to improve the function and/or extend the useful life of the heart valve without subjecting the patient to the invasiveness of open heart surgery.

A typical aortic heart valve may comprise three leaflets (i.e., a tri-leaflet valve), although two leaflet (i.e. bi-leaflet) and four leaflet valves are known to occur in a portion of the population. The devices and methods described herein are discussed with preference toward treatment of the aortic (tri-leaflet) heart valve. However, it is fully contemplated that the devices and methods described herein may be adapted for use in, the treatment of a non-aortic heart valve, an asymmetric aortic heart valve (i.e. a bi-leaflet valve), an asymmetrically-diseased or asymmetrically-calcified aortic heart valve, or other suitable uses and the like. One of ordinary skill in the art will understand that in treating a heart valve in accordance with the following disclosure, the relative orientations and directions associated with the described devices and methods may be modified to accommodate the specifics (i.e., orientation, location, size, etc.) of a particular heart valve undergoing treatment.

In some embodiments, a percutaneously-deployable medical device may be employed to repair a heart valve. A medical device may be introduced into the vasculature and advanced through a vessel (i.e., the aorta) in a retrograde direction and into a heart valve (i.e. the aortic valve) in a collapsed delivery configuration, with or without the aid of a separate delivery catheter. The medical device may then be deployed to an expanded configuration. In some embodiments, the medical device may “crack” or re-open calcified valve leaflets, and/or expand the aortic annulus, thereby restoring a portion or all of normal function and blood flow. In some embodiments, one or more of several functions or events may occur before, upon, or after deploying or expanding the medical device. The medical device may pre-dilitate the heart valve for a subsequent procedure. The medical device may also, for example, deploy a distal protection filter.

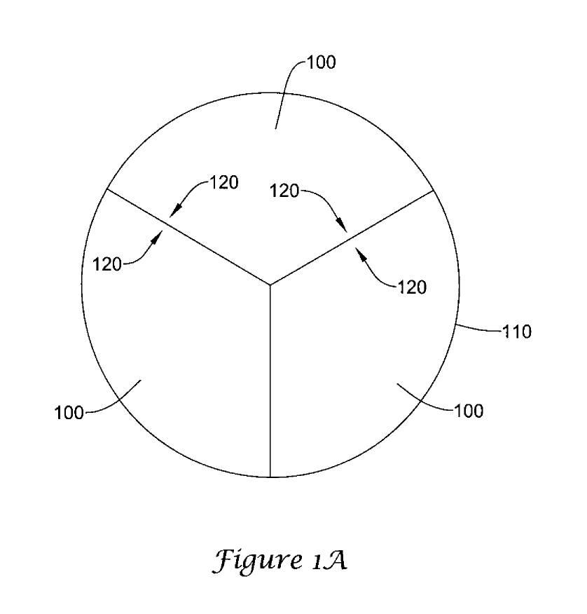

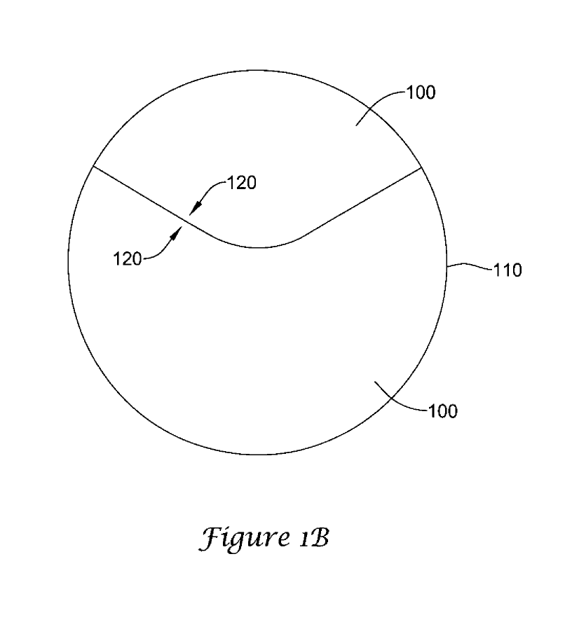

FIG. 1A schematically illustrates a partial cross-sectional view of an example tri-leaflet heart valve. Three valve leaflets 100 are fixed at their radially outermost edges (relative to an axis through the valve that would be directed perpendicularly through the view shown) to the valve annulus 110. In some cases, one of the valve leaflets 100 may be larger in size or surface area than the other two valve leaflets 100, or the three valve leaflets 100 may be substantially the same size. The valve leaflets 100 each have a free margin 120 opposite the valve annulus 110 which is free to move relative to the valve annulus 110. The free margins 120 of adjacent valve leaflets 100 come together or abut each other at a leaflet intersection when the valve is closed.

FIG. 1B schematically illustrates a partial cross-sectional view of an example bi-leaflet heart valve, which may be, for example, a non-aortic valve or an asymmetrical aortic valve. Two valve leaflets 100 are fixed at their radially outermost edges (relative to an axis through the valve that would be directed perpendicularly through the view shown) to the valve annulus 110. In some cases, one of the valve leaflets 100 may be larger in size or surface area than the second valve leaflet 100. The valve leaflets 100 each have a free margin 120 opposite the valve annulus 110 which is free to move relative to the valve annulus 110. The free margins 120 come together or abut each other at a leaflet intersection when the valve is closed.

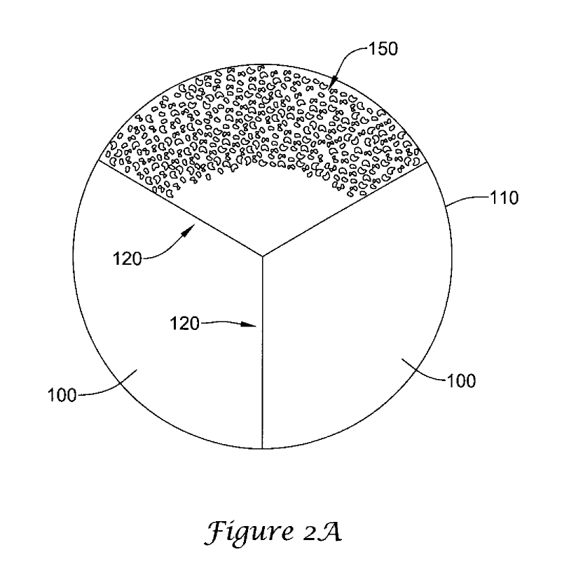

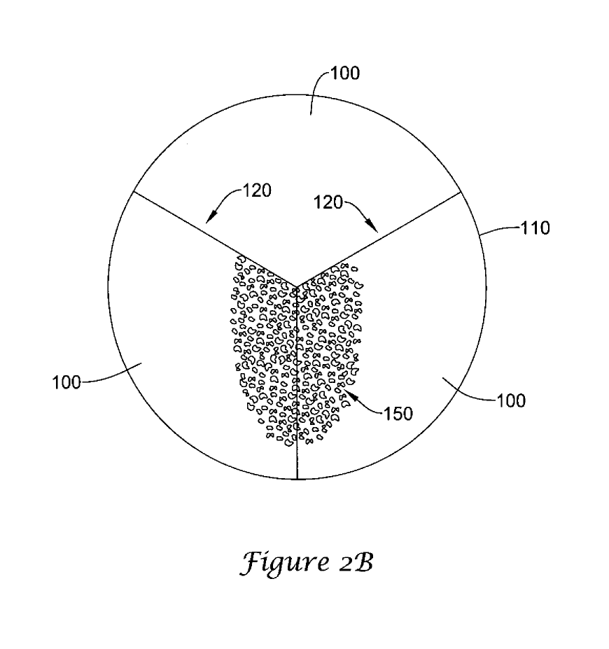

FIGS. 2A and 2B schematically illustrate a partial cross-sectional view of an example diseased tri-leaflet heart valve having asymmetric calcification 150. The valve structure is substantially similar to that described above with respect to FIG. 1B. In the examples of FIGS. 2A and 2B, one or more valve leaflets 100 may include calcification 150, thereby limiting their function. In some embodiments, the calcification 150 may bond or fuse two valve leaflets 100 together, effectively making the two valve leaflets 100 function as a single leaflet having limited flexibility and motion. In some embodiments, the calcification 150 may become so hardened that the valve leaflets 100 may exhibit an undesired, preferential opening biased away from the calcification 150. Reduced blood flow over the calcification 150 may contribute to additional calcium deposition, further exacerbating the diseased condition.

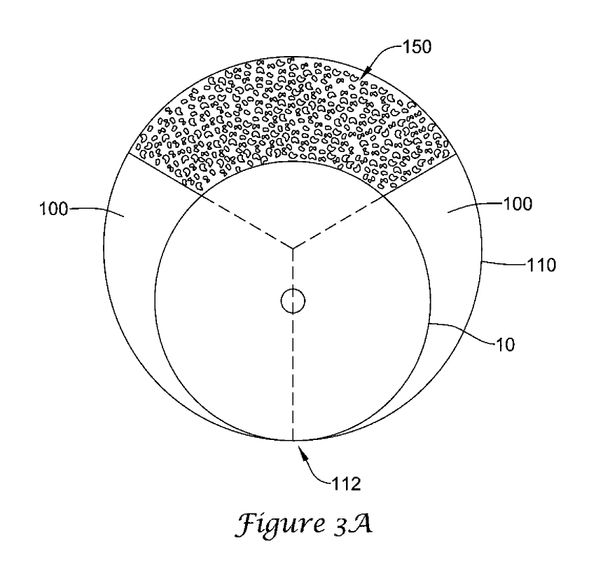

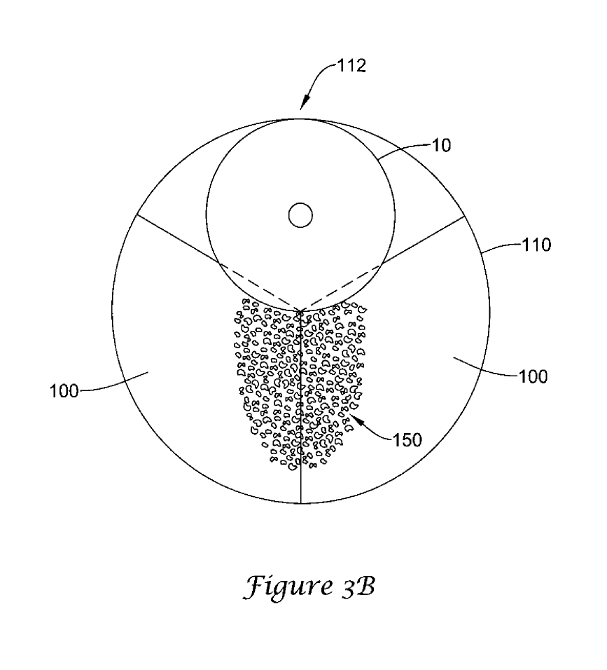

FIGS. 3A and 3B schematically illustrate a typical valvuloplasty device having a single balloon 10 disposed within the valve annulus 110. During inflation, the balloon 10 may expand in or toward an area where the balloon 10 experiences the least resistance to expansion. As can be seen in FIGS. 3A and 3B, the calcification 150 may cause the balloon 10 to expand offset from a central axis of the valve, with the balloon 10 undesirably biased toward an opposing, non-calcified or less calcified portion of the valve annulus 110. Continued inflation and an increase in the internal pressure of the balloon 10 may be required to “crack” the calcification. Increasing the pressure within the balloon 10 may also increase the pressure against the valve annulus 110, which generally becomes concentrated and highest at a single point 112 opposite the calcification 150. The concentration of pressure at point 112 may increase the risk of damage or injury to the valve annulus 110. Rupture of the valve annulus 110 may have catastrophic consequences and is highly undesirable. In order to more evenly distribute the inflation pressure against the valve annulus 110 and reduce the risk of rupture, a valvuloplasty device having a plurality of balloons has been developed.

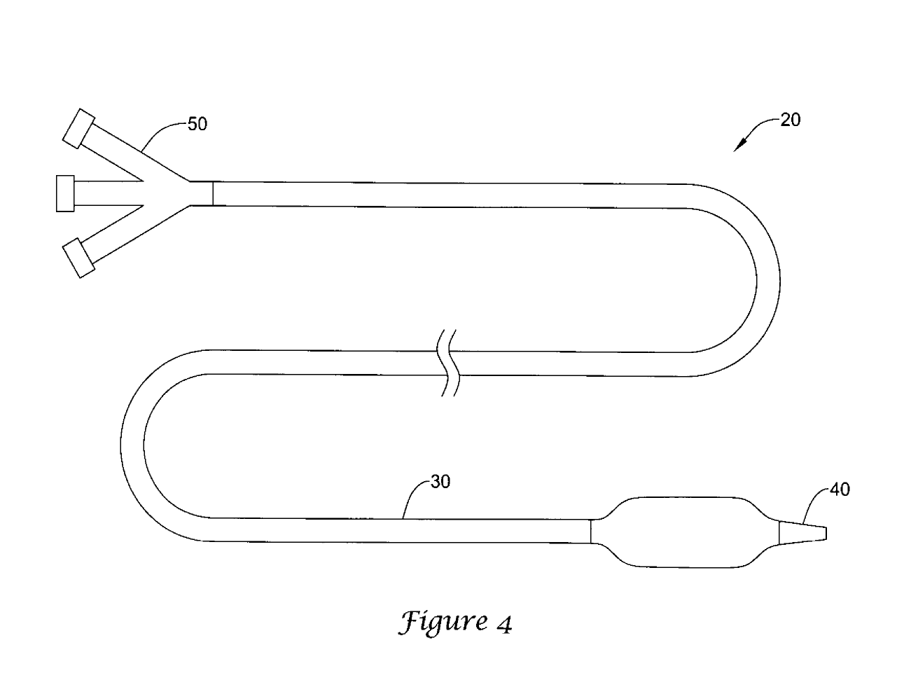

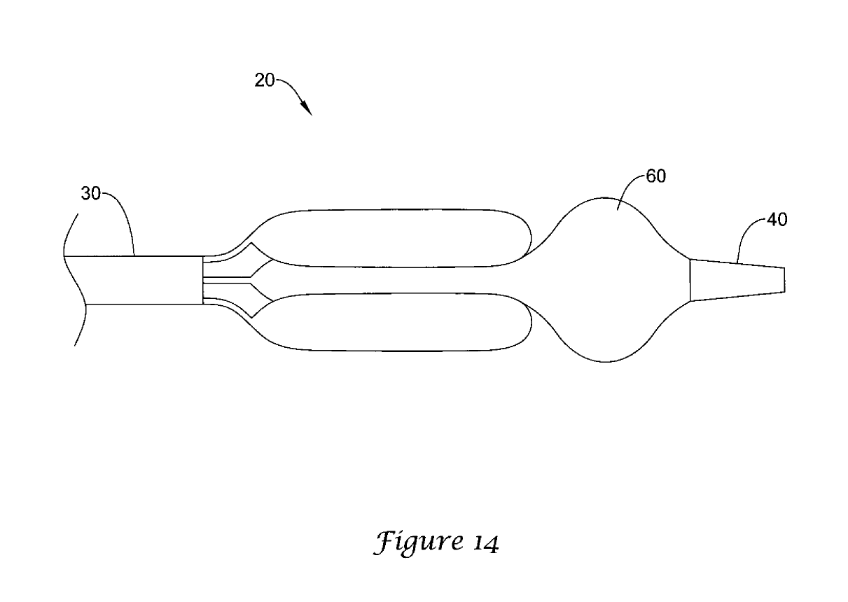

FIG. 4 schematically illustrates a medical device 20 having a plurality of elongated balloons disposed at a distal end of an elongate shaft 30. In some embodiments, the plurality of elongated balloons may be arranged in an orientation substantially parallel to a longitudinal axis of the medical device 20. In some embodiments, the plurality of balloons includes a generally cylindrical central body portion having a generally conical waist disposed at opposite ends of the central body portion. Other suitable balloon shapes, including a tapered balloon body portion and/or an hourglass-shaped balloon body portion, are also possible depending upon the desired configuration. In some embodiments, the plurality of elongated balloons may be joined together at their respective distal ends by a distal tip 40, or other suitable joining means. In some embodiments, the plurality of balloons may further include a compliant distal balloon 60, as shown, for example, in FIG. 14, which may be in addition to, or take the place of, the distal tip 40. The distal balloon 60 may form an inflatable distal tip. In some embodiments, the distal balloon 60 may be used as an anchoring means to prevent downstream migration of the medical device 20 as the plurality of balloons is inflated. In some embodiments, the distal balloon 60 may substantially block blood flow through a heart valve during a procedure, if such temporary blockage were desired.

Each of the plurality of balloons may be configured to transition between a collapsed configuration and an inflated configuration. In the collapsed configuration, the plurality of balloons may be configured to be received within a lumen or a distally-facing cavity of the elongate shaft 30, or another suitable device such as, but not limited to, a delivery sheath, a catheter, a hypotube, an endoscope, a distal protection device, and the like. In some embodiments, each of the plurality of balloons may be fluidly connected to a manifold 50 at a proximal end of the medical device 20 by an inflation lumen (not shown) disposed within the elongate shaft 30. The manifold 50 may be fluidly connected to one or more sources of inflation fluid. In some embodiments, two or more inflation lumens may be fluidly connected, such that two or more balloons may share a common source of inflation fluid and/or transition between the collapsed configuration and the inflated configuration substantially simultaneously. Alternatively, two or more balloons may be fluidly connected to a single inflation lumen (not shown). In other words, in some embodiments, two or more balloons may be configured to inflate and/or deflate together simultaneously, in sequence, or in another order, grouping, or relationship, as desired.

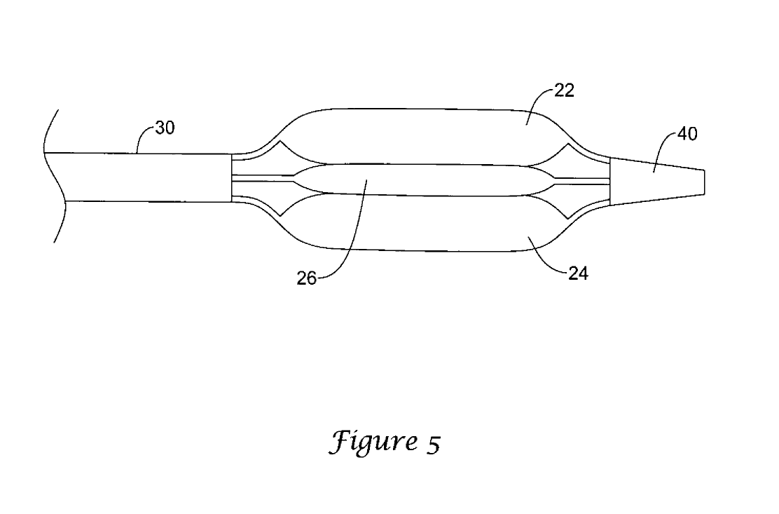

In some embodiments, the plurality of elongated balloons may include a first balloon 22, a second balloon 24, and a third balloon 26, as shown, for example, in FIG. 5. In some embodiments, one or more of the first balloon 22, the second balloon 24, and the third balloon 26 may include a generally conical proximal waist, a generally conical distal waist, and a generally uniform diameter body portion disposed between the proximal waist and the distal waist. In some embodiments, the plurality of balloons may be configured for placement at least partially within the valve annulus 110 such that the distal tip 40 is disposed upstream of the valve leaflets 100. For example, the body portion of the plurality of balloons may be disposed within the valve annulus 110, with the distal waist disposed within the left ventricle and the proximal waist disposed within the aortic arch, although the exact placement may vary as needed or desired. In some embodiments, one or more of the first balloon 22, the second balloon 24, and the third balloon 26 may include a lumen passing longitudinally therethrough for the passage of a guidewire or other device. Alternatively, a separate shaft or lumen may extend past the plurality of balloons, for example, centrally disposed along the longitudinal axis of the medical device 20, for the passage of a guidewire or other device. In some embodiments, a lumen for the passage of a guidewire or other device may extend through the distal tip 40.

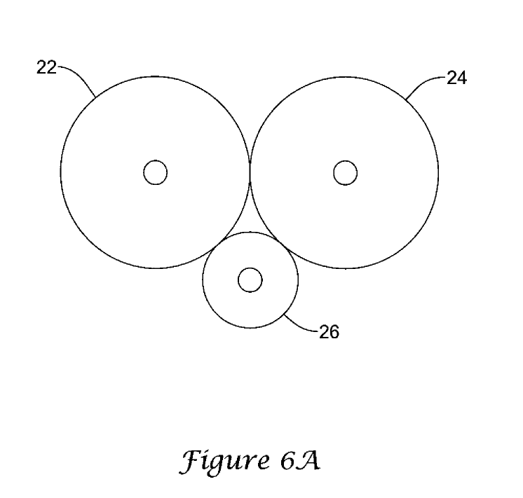

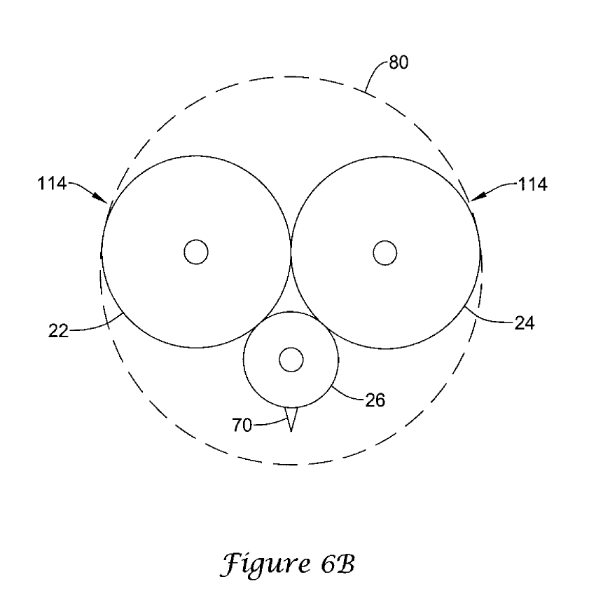

In some embodiments, the first balloon 22 and the second balloon 24 may be sized and configured substantially similarly, or they may be sized differently as desired. In general, the third balloon 26 may include a body portion having a first diameter. The first balloon 22 and the second balloon 24 may each include a body portion having a second diameter, wherein the second diameter may be about 1.25, 1.5, 1.75, 2.0, 2.25, 2.5, 2.75, 3.0, or more, times greater than the first diameter. In other words, the first balloon 22 and the second balloon 24 may be generally larger in size/diameter than the third balloon 26, as seen schematically in FIG. 6A. In some embodiments, the third balloon 26 may be inflated to a higher inflation pressure than the first balloon 22 and/or the second balloon 24.

In some embodiments, a cutting element 70 may be disposed on an outer surface of the third balloon 26, as shown schematically in FIG. 6B. In some embodiments, the cutting element 70 may be oriented longitudinally and/or substantially parallel to the longitudinal axis of the medical device 20. In some embodiments, the cutting element 70 may comprise a single cutting element or a plurality of cutting elements. In some embodiments, the cutting element 70 may comprise a conventional cutting blade or a microscalpel blade. As used herein, a “microscalpel blade” is meant to indicate any cutting blade, surface, element, edge, or the like that comprises a cutting edge or surface that is generally smaller and/or sharper than a conventional cutting blade. Generally speaking, microscalpel blades have a cutting edge or surface that is at least 2 to 10 times sharper than the cutting edge of a conventional cutting blade, and may include a cutting edge having a radius of curvature less than about 100 nm, less than about 50 nm, or less than about 10 nm. In some embodiments, the cutting element 70 may include an optional coating thereon (not shown) to, for example, enhance strength, increase lubricity, impart radiopacity, deliver therapeutic agents, and the like.

Furthermore, as shown schematically in FIG. 6B, the first balloon 22, the second balloon 24, and the third balloon 26, along with the cutting element 70, may be sized such that in the inflated configuration, each of the elements is positioned within a single reference feature 80, designated by a phantom-line circle disposed about the first balloon 22, the second balloon 24, the third balloon 26, and the cutting element 70. In general, the reference feature 80 may correspond in size to the valve annulus 110. Accordingly, when each of the plurality of balloons is fully inflated to the inflated configuration, each of the balloons, as well as the cutting element 70, may generally not extend beyond the valve annulus 110 in which they are disposed. Such an arrangement may prevent the cutting element 70 from cutting through, weakening, or otherwise injuring the valve annulus 110 compared to a system using three balloons that are all substantially the same size.

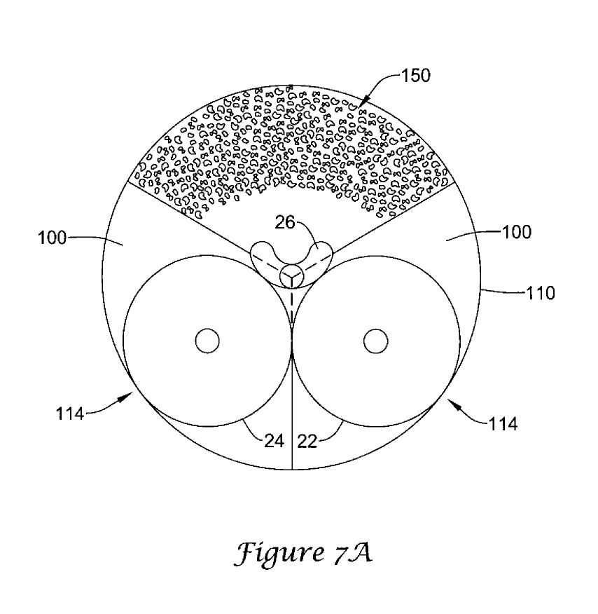

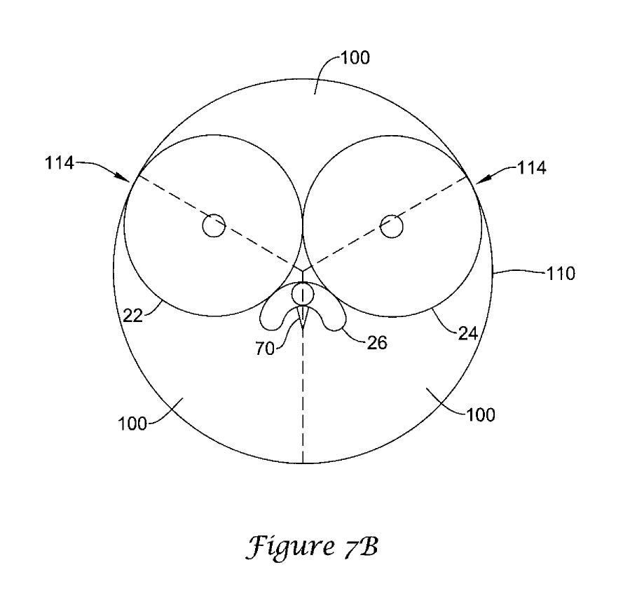

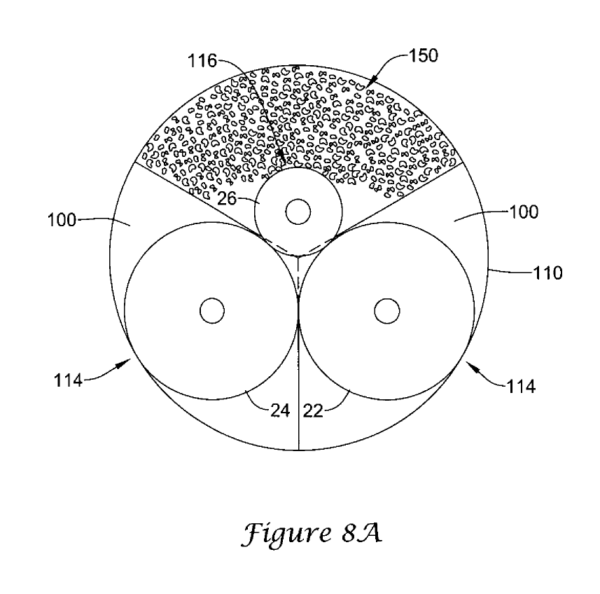

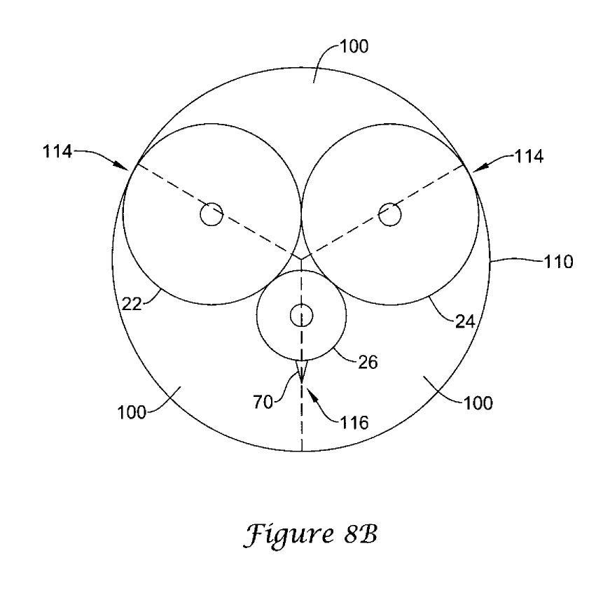

FIGS. 7A and 8A schematically illustrate using an example medical device 20 such as that shown in FIGS. 5 and 6A to treat the condition shown in FIG. 2A. In use, the medical device 20 may be advanced through the vasculature in a retrograde direction through a vessel (i.e., the aorta) to a treatment site (i.e., the aortic valve), which may have asymmetric calcification 150. The distal tip 40 may be positioned upstream or distal of the treatment site such that the plurality of balloons is positioned within the valve annulus 110 of the treatment site. Next, inflation fluid is introduced into the first balloon 22 and the second balloon 24, thereby expanding the balloons from the collapsed configuration to the inflated configuration. In the inflated configuration, the first balloon 22 and the second balloon 24 may have a first inflation pressure therein. As the inflation fluid is introduced to the first balloon 22 and the second balloon 24, the first balloon 22 and the second balloon 24 may expand within the valve annulus 110, biased away from the most diseased portion of the valve leaflets 100 toward a non-diseased or less-diseased portion of the valve leaflets 100. Generally, the first balloon 22 and the second balloon 24 will each form a point of contact 114 with the valve annulus 110, thereby distributing an outwardly-exerted force against the valve annulus 110 more evenly than a single balloon. The first balloon 22 and the second balloon 24, arranged in this manner, will orient the third balloon 26 toward the most diseased portion of the valve leaflets 100, where it will be most effective in treating asymmetric calcification 150. The third balloon 26 is then expanded to the inflated configuration, wherein the third balloon achieves a second inflation pressure therein. In some embodiments, the second inflation pressure may be greater than the first inflation pressure. In some embodiments, the second inflation pressure may be 1.25, 1.5, 1.75, 2.0, 2.25, 2.5, 2.75, 3.0, or more, times greater than the first inflation pressure. The third balloon 26 may form a point of contact 116 with the asymmetric calcification 150, where the increased pressure of the third balloon 26 relative to the first balloon 22 and the second balloon 24 may “crack”, or force open, the asymmetric calcification. Since the first balloon 22 and the second balloon 24 each form a point of contact 114 with the valve annulus 110, an outwardly-exerted force against the valve annulus 110 resulting from increasing the inflation pressure within the third balloon 26 is distributed to more than one contact point, thereby reducing the risk of injury to the valve annulus 110, while presenting an improved outer profile of the medical device 20 within the valve annulus 110, compared to a single balloon 10. Additionally, spaces or gaps between the first balloon 22, the second balloon 24, and the third balloon 26 may permit perfusion blood flow through the heart valve around the plurality of balloons during the procedure, thereby reducing the pressure gradient between the left ventricle and the aorta and also reduce the need for fast pacing, or pulsing the heart at high frequency, due to obstruction of blood flow from the heart to the aorta. After cracking or opening the asymmetric calcification 150, the plurality of balloons may be deflated to the collapsed configuration. In some embodiments, the first balloon 22, the second balloon 24, and the third balloon 26 may be deflated simultaneously, one-at-a-time in sequence, or some combination thereof, as desired. The plurality of balloons may be retracted within the elongate shaft 30 and withdrawn from the vasculature. In some embodiments, the plurality of balloons may be withdrawn from the vasculature without retracting the plurality of balloons into the elongate shaft, or the plurality of balloons may be disposed within a separate retrieval sheath.

FIGS. 7B and 8B schematically illustrate using an example medical device 20 such as that shown in FIGS. 5 and 6B to treat the conditions shown in FIGS. 1B and 2B. In use, the medical device 20 may be advanced through the vasculature in a retrograde direction through a vessel (i.e., the aorta) to a treatment site (i.e., the aortic valve), which may have asymmetric calcification 150. The distal tip 40 may be positioned upstream or distal of the treatment site such that the plurality of balloons is positioned within the valve annulus 110 of the treatment site. Next, inflation fluid is introduced into the first balloon 22 and the second balloon 24, thereby expanding the balloons from the collapsed configuration to the inflated configuration. In the inflated configuration, the first balloon 22 and the second balloon 24 may have a first inflation pressure therein. As the inflation fluid is introduced to the first balloon 22 and the second balloon 24, the first balloon 22 and the second balloon 24 may expand within the valve annulus 110, biased away from the most diseased portion of the valve leaflets 100 toward a non-diseased or less-diseased portion of the valve leaflets 100. Generally, the first balloon 22 and the second balloon 24 will each form a point of contact 114 with the valve annulus 110, thereby distributing an outwardly-exerted force against the valve annulus 110 more evenly than a single balloon. The first balloon 22 and the second balloon 24, arranged in this manner, will orient the third balloon 26 with a cutting element 70 disposed thereon toward the most diseased portion of the valve leaflets 100, where it will be most effective in treating a bi-leaflet valve (as in FIG. 1B) and/or asymmetric calcification 150 (as in FIG. 2B). The third balloon 26 is then expanded to the inflated configuration, wherein the third balloon 26 achieves a second inflation pressure therein. In some embodiments, the second inflation pressure may be greater than the first inflation pressure. In some embodiments, the second inflation pressure may be 1.25, 1.5, 1.75, 2.0, 2.25, 2.5, 2.75, 3.0, or more, times greater than the first inflation pressure. The third balloon 26 (and the cutting element 70 thereon) may form a point of contact 116 with the leaflet 100 and/or asymmetric calcification 150, where the increased pressure of the third balloon 26 relative to the first balloon 22 and the second balloon 24 along with the cutting element 70 may cut one valve leaflet 100 into two valve leaflets 100 (as in FIG. 1B), and/or the cutting element 70 may cut through the asymmetric calcification 150 along the leaflet intersection to separate two valve leaflets 100 (as in FIG. 2B), thereby at least partially restoring normal function of the leaflets 100 and/or the heart valve. As discussed earlier, the plurality of balloons may be sized such that the cutting element 70 is prevented from cutting or damaging the valve annulus 110. The second inflation pressure may provide increased support for the cutting element 70, thereby increasing the force the cutting element 70 may exert upon the asymmetric calcification 150 and permitting the cutting element 70 to treat extremely tough or rigid calcification. Since the first balloon 22 and the second balloon 24 each form a point of contact 114 with the valve annulus 110, an outwardly-exerted force against the valve annulus 110 resulting from increasing the inflation pressure within the third balloon 26 is distributed to more than one contact point, thereby reducing the risk of injury to the valve annulus 110, while presenting an improved outer profile of the medical device 20 within the valve annulus 110, compared to a single balloon 10. Additionally, spaces or gaps between the first balloon 22, the second balloon 24, and the third balloon 26 may permit perfusion blood flow through the heart valve around the plurality of balloons during the procedure, thereby reducing the pressure gradient between the left ventricle and the aorta and also reduce the need for fast pacing, or pulsing the heart at high frequency, due to obstruction of blood flow from the heart to the aorta. After cutting the leaflet 100 and/or the asymmetric calcification 150, the plurality of balloons may be deflated to the collapsed configuration. In some embodiments, the first balloon 22, the second balloon 24, and the third balloon 26 may be deflated simultaneously, one-at-a-time in sequence, or some combination thereof, as desired. The plurality of balloons may be retracted within the elongate shaft 30 and withdrawn from the vasculature. In some embodiments, the plurality of balloons may be withdrawn from the vasculature without retracting the plurality of balloons into the elongate shaft, or the plurality of balloons may be disposed within a separate retrieval sheath.

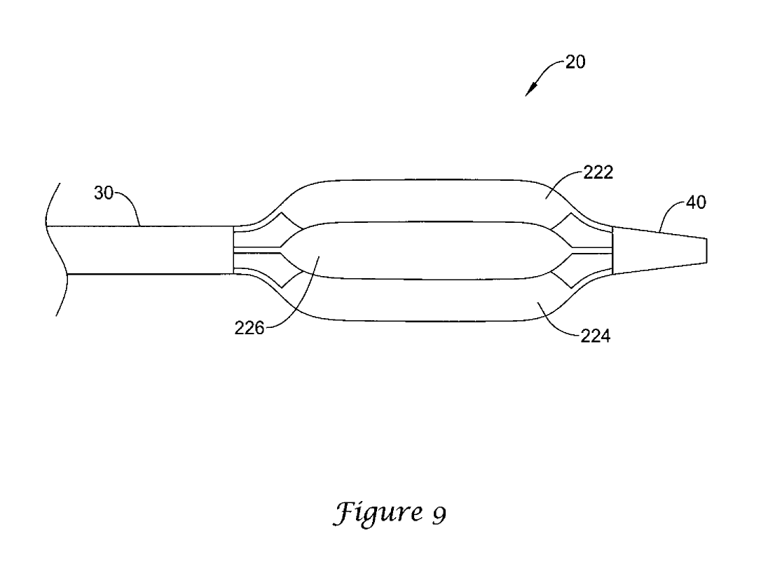

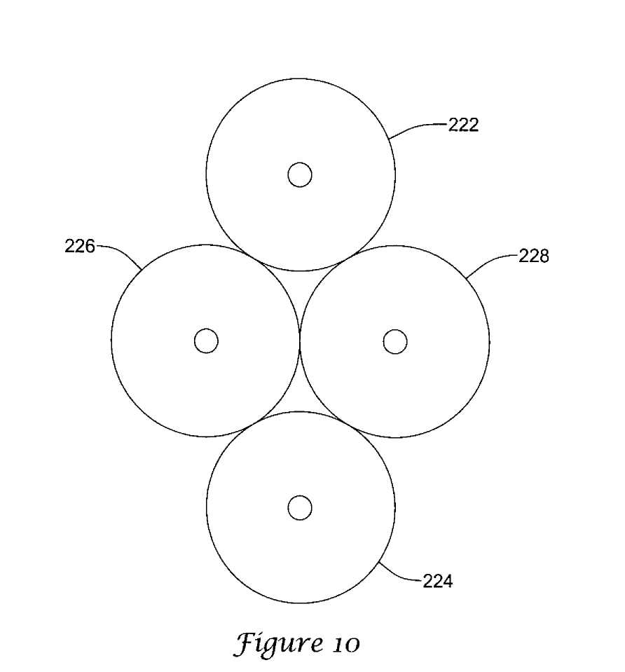

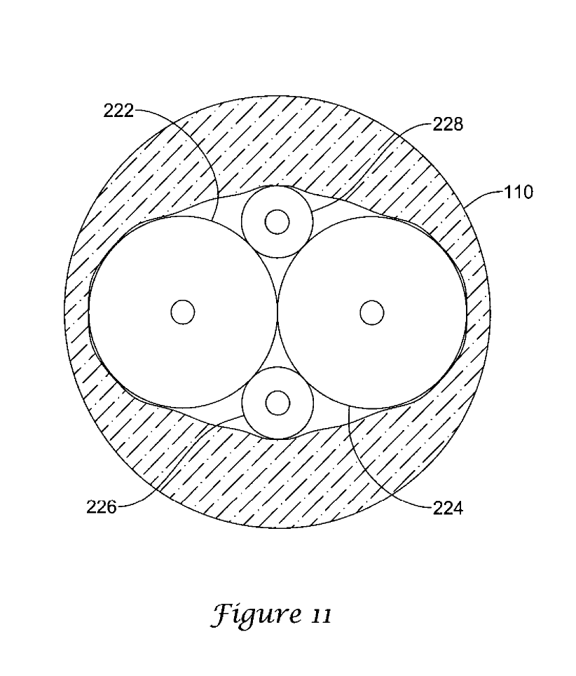

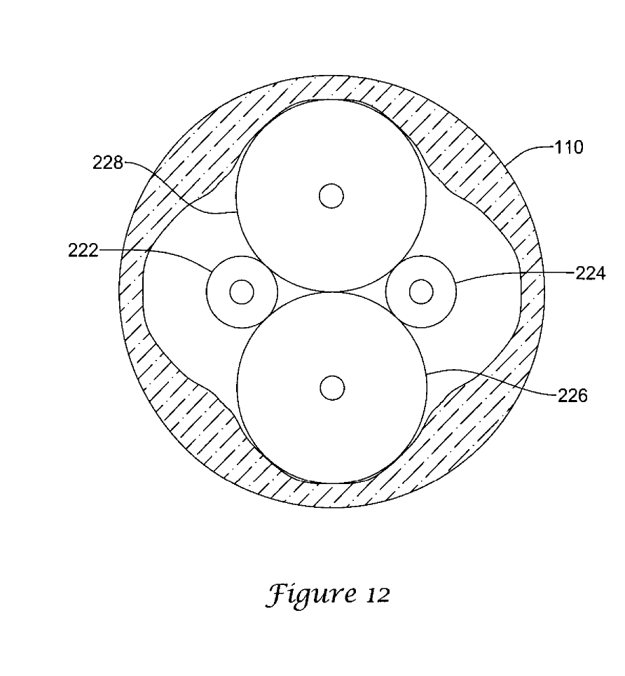

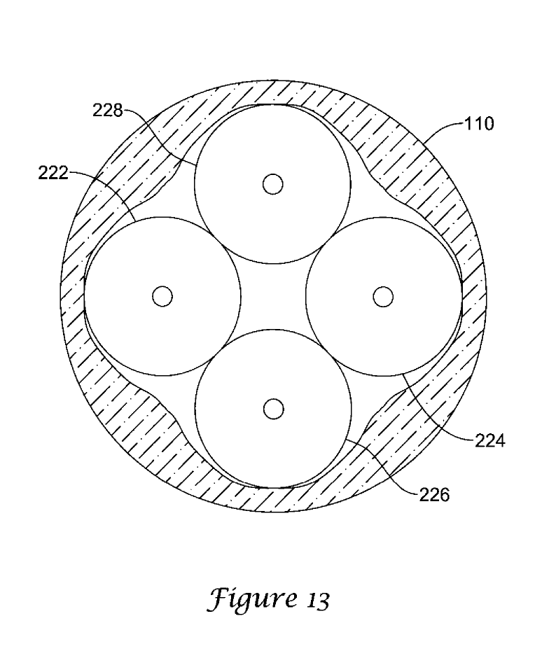

In some embodiments, the plurality of elongated balloons may include a first balloon 222, a second balloon 224, a third balloon 226, and a fourth balloon 228, as shown schematically in FIGS. 9-10. The medical device 20 may be useful in treating conditions similar to those described above, as well as calcification or stenosis deposited on the walls of a vessel lumen. The first balloon 222, the second balloon 224, the third balloon 226, and the fourth balloon 228 may cooperate to actuate between a collapsed configuration and an inflated configuration in opposing pairs so as to present a non-circular profile for expanding the valve annulus 110 and/or the vessel lumen. In use, the first balloon 222 and the second balloon 224 may be inflated together, as seen schematically in FIG. 11. Next, the first balloon 222 and the second balloon 224 may be deflated together while at the same time the third balloon 226 and the fourth balloon 228 are inflated together, as seen schematically in FIG. 12, thereby maintaining a minimum inner diameter of the valve annulus 110 and/or the vessel lumen. The first balloon 222 and the second balloon 224 may work as a first opposing pair, and the third balloon 226 and the fourth balloon 228 may work as a second opposing pair, the first and second pairs alternating between the collapsed and inflated configurations as necessary to obtain a desired amount of opening within the valve annulus 110 and/or the vessel lumen. The inflation and deflation of the first and second pairs may repeat as needed. At approximately the mid-point of each cycle between alternating first and second pairs, a configuration such as that shown schematically in FIG. 13 will be achieved, where each of the four balloons has approximately the same outer diameter or cross-section while partially inflated. Similarly, after the first and second pairs have alternated between the collapsed and inflated configurations to achieve the desired amount of opening within the valve annulus 110 and/or the vessel lumen, all four balloons may be inflated to substantially the same size and pressure so as to regularize (to restore to a generally circular opening) the form or shape of the valve annulus 110 and/or the vessel lumen. Spaces or gaps between the first balloon 222, the second balloon 224, the third balloon 226, and the fourth balloon 228 may permit perfusion blood flow through the heart valve around the plurality of balloons during the procedure, thereby reducing the pressure gradient between the left ventricle and the aorta and also reduce the need for fast pacing, or pulsing the heart at high frequency, due to obstruction of blood flow from the heart to the aorta. After opening the valve annulus 110 and/or the vessel lumen, the plurality of balloons may be deflated to the collapsed configuration. In some embodiments, the first balloon 222, the second balloon 224, the third balloon 226, and the fourth balloon 228 may be deflated simultaneously, one-at-a-time in sequence, or some combination thereof, as desired. The plurality of balloons may be retracted within the elongate shaft 30 and withdrawn from the vasculature. In some embodiments, the plurality of balloons may be withdrawn from the vasculature without retracting the plurality of balloons into the elongate shaft, or the plurality of balloons may be disposed within a separate retrieval sheath.

The plurality of balloons as described herein may be made of any suitable material, for example, a polymeric material, a thin-film metal or metal alloy, a metal-polymer composite, combinations thereof, and the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials.

The elongate shaft 30 and/or the distal tip 40, along with other suitable components of the medical device 20, may be made from materials such as metals, metal alloys, polymers, metal-polymer composites, or other suitable materials, and the like. In some embodiments, the elongate shaft 30 and the distal tip 40 are made from the same material, although this is not required. Some examples of some suitable materials may include metallic materials and/or alloys such as stainless steel (e.g. 304v stainless steel or 316L stainless steel), nickel-titanium alloy (e.g., nitinol, such as super elastic or linear elastic nitinol), nickel-chromium alloy, nickel-chromium-iron alloy, cobalt alloy, nickel, titanium, platinum, or alternatively, a polymer material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some embodiments, portions of the medical device 20 may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. Suitable materials can include, but are not limited to, bismuth subcarbonate, iodine, gold, platinum, palladium, tantalum, tungsten or tungsten alloy, and the like.

In some embodiments, the medical device 20 may be re-used in a subsequent procedure. In some embodiments, the plurality of balloons may be withdrawn through the elongate shaft 30 prior to performing a subsequent procedure and the elongate shaft 30 may be used as a delivery sheath for another medical device to be used in the subsequent procedure.

Although not expressly illustrated, a portion of the medical device 20 and/or the elongate shaft 30 may be configured to include a predetermined bending configuration aligning with the curve of the aorta and/or the aortic arch. For example, in some embodiments, the elongate shaft 30 may include a directional bending component (not shown) that aligns the elongate shaft 30 and/or a separate delivery sheath, if included, with the curve of the aorta and/or the aortic arch. For example, the elongate shaft 30 may include a metallic wire or strip (not shown) embedded within a wall of the elongate shaft 30 or disposed within a lumen within the wall of the elongate shaft 30. The metallic wire or strip may be flattened or otherwise configured to have a predetermined or preferential bending direction. As the elongate shaft 30 is advanced through the aortic arch, the elongate shaft 30 may align such that the plurality of balloons will assume a predetermined orientation within the treatment site (i.e., the valve annulus 110). In some embodiments, the predetermined orientation may correspond to certain desired valve leaflets 100 or certain desired leaflet intersection(s) between the valve leaflets 100.

It should be understood that although the above discussion was focused on a medical device and methods of use within the coronary vascular system of a patient, other embodiments of medical devices or methods in accordance with the invention can be adapted and configured for use in other parts of the anatomy of a patient. For example, devices and methods in accordance with the invention can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the medical devices described herein with respect to percutaneous deployment may be used in other types of surgical procedures as appropriate. For example, in some embodiments, the medical devices may be deployed in a non-percutaneous procedure, including an open heart procedure. Devices and methods in accordance with the invention can also be adapted and configured for other uses within the anatomy.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

We claim:

1. A medical device for valvuloplasty of a heart valve, comprising:
an elongate shaft having a plurality of elongated balloons disposed at a distal end thereof, each elongated balloon including a proximal end and a distal end;
wherein the plurality of elongated balloons further comprises a first balloon, a second balloon, and a third balloon;
a manifold disposed at a proximal end of the elongate shaft, the manifold in fluid communication with the plurality of elongated balloons; and
a distal tip disposed distal of the plurality of elongated balloons, the distal end of each elongated balloon being joined together by the distal tip;
wherein the plurality of elongated balloons is configured to selectively expand from a collapsed configuration to an inflated configuration.

2. The medical device of claim 1, wherein each of the plurality of elongated balloons includes a generally cylindrical body portion.

3. The medical device of claim 2, wherein the plurality of elongated balloons is oriented substantially parallel to a central longitudinal axis of the medical device.

4. The medical device of claim 1, wherein the elongate shaft is configured to receive the plurality of elongated balloons therein in the collapsed configuration.

5. The medical device of claim 2, wherein the first balloon and the second balloon each have a maximum outer diameter that is greater than a maximum outer diameter of the third balloon.

6. The medical device of claim 1, wherein the first balloon and the second balloon each have a lower inflation pressure in the inflated configuration than the third balloon.

7. The medical device of claim 1, further including a lumen extending through the distal tip.

8. The medical device of claim 1, wherein the third balloon includes a cutting element disposed thereon.

9. The medical device of claim 1, wherein the distal tip includes a distal balloon.

10. A medical device for valvuloplasty of a heart valve, comprising:
an elongate shaft having a plurality of elongated balloons disposed at a distal end thereof, each elongated balloon including a proximal end and a distal end;
wherein the plurality of elongated balloons further comprises a first balloon and a second balloon arranged as a first opposing pair, and a third balloon and a fourth balloon arranged as a second opposing pair;
a manifold disposed at a proximal end of the elongate shaft, the manifold in fluid communication with the plurality of elongated balloons; and
a distal tip disposed distal of the plurality of elongated balloons, the distal end of each elongated balloon being joined together by the distal tip;
wherein each of the plurality of elongated balloons is configured to selectively actuate between a collapsed configuration and an inflated configuration.

11. The medical device of claim 10, wherein the first balloon and the second balloon are configured to selectively actuate between the collapsed configuration and the inflated configuration substantially simultaneously.

12. The medical device of claim 11, wherein the third balloon and the fourth balloon are configured to selectively actuate between the collapsed configuration and the inflated configuration substantially simultaneously.

13. The medical device of claim 12, wherein the first opposing pair and the second opposing pair are configured to selectively actuate between the collapsed configuration and the inflated configuration substantially simultaneously and in opposite directions.

14. The medical device of claim 10, wherein the elongate shaft is configured to receive the plurality of elongated balloons therein in the collapsed configuration.

15. The medical device of claim 10, further including a lumen extending through the distal tip.

16. The medical device of claim 1, wherein the distal tip includes a distal balloon.

17. The medical device of claim 10, wherein each of the plurality of elongated balloons includes a generally cylindrical body portion.

18. The medical device of claim 17, wherein the plurality of elongated balloons is oriented substantially parallel to a central longitudinal axis of the medical device.
